(19) United States
(12) Patent Application Publication
(10) Pub. No.: US 2021/0121174 A1
(43) Pub. Date: Apr. 29, 2021

(54) SURGICAL STAPLER, SURGICAL STAPLE ARRANGEMENT AND METHOD FOR APPROXIMATION OF MUSCULAR OR APONEUROTIC STRUCTURES

(71) Applicant: Diogo de Freitas Valeiro GARCIA, São Paulo (BR)

(72) Inventor: Diogo de Freitas Valeiro GARCIA, São Paulo (BR)

(21) Appl. No.: 16/617,421

(22) PCT Filed: Jun. 11, 2018
(86) PCT No.: PCT/BR2018/050186

§ 371 Date: Nov. 26, 2019

(30) Foreign Application Priority Data
Jun. 9, 2017 (BR) 102017012397-9

(51) Int. Cl. A61B 17/068

(52) U.S. Cl. CPC A61B 17/068; A61B 2017/00004

(57) ABSTRACT

The present invention describes a surgical stapler, a surgical staple arrangement and a method for approximate of muscular or aponeurotic structures. More precisely, the surgical staple comprises stapling structures associated to an alignment structure, in one of its embodiments the stapling structures holds at least a staple arrangement, approximating it rectilinearly clamping the staple arrangement, the surgical staple arrangement comprises a male and a female portion comprising a plurality of clamping structures that associate to each other and the method discloses steps for approximating muscular or aponeurotic structures and keep them together by the usage of and staple arrangement. The present invention is situated in the field of medical science, medical device and surgical stapler.

### TECHNICAL FIELD

The present invention describes a surgical stapler, a surgical staple arrangement and a method for the approximation of muscular or aponeurotic structures. The present invention is situated in the field of medical science, medical device and surgical stapler.

### BACKGROUND TECHNIQUE

In surgical cases where the opening of the aponeurosis is performed, as exploratory laparotomy and hernias corrections, or in cases of Diastasis of the rectus abdominis muscle, there is the need to put the aponeurosis back together, where such procedure is made by manual suture, which requires experience and time wasted.

In such examples as diastasis of the rectus abdominis muscle, or also known as abdominal diastasis, is basically due to a stretching of the abdominal muscle where there is an opening or a visible bulge in the region of the belly, i.e., the abdominal muscles are displaced laterally into the patient's belly. This clinical occurrence is very common in women who go through gestations, since with the dilation of the uterus the muscles end up being displaced laterally. Further, there are other forms of abdominal diastasis, for example in cases of obesity, malnutrition, some type of intense abdominal exercise, due to the presence of tumors, etc.

By being noticeably remarkable, abdominal diastasis may generate some discomfort in a patient, as deformities may occur in the belly region, which may discourage them psychologically, causing low estimates of their physical appearance. For this reason, many patients are lead to surgical procedures for these aesthetic corrections. In addition, there are still patients who need this procedure to treat some type of disease, that is, not only for aesthetic purposes.

The present ways of performing surgical procedure regarding closure of abdominal muscles are based on highly invasive means where it is necessary to open the entire abdominal region so that it is possible to reach the muscles and approach them again. In these procedures, as soon as the surgeon approaches the patient's muscles, he performs the suture by means of a specific wire, fixing the aponeurosis of the abdominal muscles.

As can be clearly perceived, the method currently used, besides being completely invasive, can generate a high risk of infection for a patient, who often undergoes this surgery simply for aesthetic purposes, and can generate several problems due to the postoperative period and, even, death. In addition, this current procedure generates unpleasant conditions to the patients after the surgeries, impacting on their recovery.

A search in scientific and patent literature pointed relevant documents for the present invention, which is described below.

The document US20020065534 discloses a stapler for muscular approximation. The stapler disclosed comprises a plier structure, where a first jaw holds a first fastening member and a second jaw holds a second fastening member, and when the jaws are close they attach the first fastening to the second fastening. However, during an medical procedure where there are need long jaws, e.g. aponeurosis procedures, the plier structure need a wider aperture of the device which implies limitations for the use of the device, since the plier structure also creates a difference of clamping force, caused by the momentum generated in the hinge.

The document WO2003088845 discloses a surgical tissue stapler that comprises two jaws, a first jaw holds a staple cartridge and a driver mechanism, a second jaw comprises an anvil. For stapling the tissue, the stapler is closed on the tissue and the driver mechanism is actuated, forcing the staples against the anvil, causing its deformation. However, for clamping procedure the staples need to deform plastically, so the thickness of the staple inflicts directly in the force that must be applied in the procedure. In another point, the driver mechanism is housed in the jaw, where in this meaning the jaw must comprise de double length of the driver mechanism to operate correctly. This fact generates an inconvenient in longer lengths. Notwithstanding, the actuation of the driver mechanism is made by applying a force on a lateral handle, causing a momentum that can make the device tremble during the procedure, resulting in injury or malfunction.

The document U.S. Pat. No. 7,442,201 discloses a fastener for use in fundoplication procedures. The fastener comprises a male and a female portion, which can be associated to each other. The male portion comprises a pair of male members associated to a rigid structure and the female portion comprises a rigid structure with a pair of holes to receive the male members. The disclosed fastener has a specific usage, wherein the rigid structure that associate de male members do not allow the staple to bend, so if it is applied in body parts that is movable, e.g. abdominal muscles, it probably cause injury or detach after the movement and also causing a discomfort to the patient.

As can be inferred from literature, there are no documents suggesting or anticipating the teachings of the present invention, so that the solution proposed here has novelty and inventive step outside the state of the art.

### SUMMARY

The present invention provides a surgical stapler, a surgical staple arrangement and a method for approximation of muscular or aponeurotic structures of the abdominal wall. The present invention is able to approximate muscular or aponeurotic structures and to associate them using a staple arrangement. The disclosed surgical staple arrangement is capable of associating muscular or aponeurotic structures, allowing the union to bend, wherein said method discloses steps for the approximation of muscular or aponeurotic structures using a surgical stapler and a staple arrangement.

In a first aspect, the present invention discloses a surgical stapler comprising stapling structures associated to an alignment structure.

In a second aspect, the present invention discloses a surgical staple arrangement comprising at least a first part and a second part associable to each other comprising a plurality of clamping structure.

In an embodiment, the present invention also provides a method for stapling muscular structures and aponeurosis comprising the steps performed by a surgical stapler as defined in the detailed description and a surgical staple arrangement as defined in the detailed description.

In a third aspect, the present invention discloses a method for approximation of muscular or aponeurotic structures of the abdominal wall comprising the steps of:
a. locate at least one stapling structure in a muscular or aponeurotic structure of the abdominal wall;
b. approximation of muscular or aponeurotic structures of the abdominal wall;
c. stapling of the muscular or aponeurotic structures of the abdominal wall.

These and other aspects of the invention will be immediately appreciated by the well versed in the art, and for companies with interests in the product segment and will be described in sufficient detail to be reproduced in the following description.

### BRIEF DESCRIPTION OF THE DRAWINGS

This invention presents the following drawings in order to define an exemplary embodiment of the proposed solution, however, without limiting the scope of protection.

FIG. 1 shows a perspective view of an embodiment of the present surgical stapler.

FIG. 2 shows a perspective view of another embodiment of the present surgical stapler.

FIG. 3 shows a perspective view of another embodiment of the present surgical stapler.

FIG. 4 shows a drawing of another embodiment of the present surgical stapler.

FIG. 5 shows a drawing of the embodiment of FIG. 4 in another position.

FIG. 6 shows an exploded view of an embodiment of the present surgical stapler.

FIG. 7 shows a perspective view of an embodiment of a stapling structure (1).

FIG. 8 shows a cut view of the staple structure shown in FIG. 7.

FIG. 9 shows a perspective view of an embodiment of a staple structure.

FIGS. 10 to 24 show a sequence of usage of an embodiment of the surgical stapler.

FIG. 25 shows an embodiment of a staple driver (6).

FIGS. 26 to 65 show different embodiments of the staple arrangements (4) as described in present invention.

FIGS. 67 to 68 show different embodiment of the connector (10) of the present invention.

### DETAILED DESCRIPTION

In a first aspect, the present invention describes a surgical stapler comprising stapling structures associated to an alignment structure. This arrangement of the surgical stapler allows a stapling of a part of a patient's body in such a way that said stapling structures are aligned to each other by the alignment structure.

The stapling structure can be any structure capable of stapling at least one staple. In an embodiment, the stapling structure is also capable to house at least one staple, where said stapling structure can be hollow or solid. In an embodiment, the stapling structure comprises projected dimension in order to promote the stapling procedure over the extension of the patient's body to be stapled. The stapling structures are associated to an alignment structure that ensures the alignment of the stapling structures, so that the stapling occurs correctly.

In one embodiment, at least one stapling structure comprises a movement along the alignment structure. The movement of the stapling structure allows approximating and distancing the stapling structures to each other. In an embodiment, the stapling structure is rectilinearly movable along the alignment structure. In an embodiment, this arrangement promotes a displacement of at least one stapling structure aligned to another stapling structure, where said movement is performed manually or automatically, and furthermore this movement is performed by means of a mechanism or directed operated by hand. In an embodiment, at least one stapling structure comprises parallel movement in relation to another stapling structure.

Moreover, in an embodiment, the stapling structures comprise at least one rip, in which said rip can be comprised in only one, in some or in all of the stapling structures. The rip can comprise any geometry, and each rip can comprise a format independently to the others. In an embodiment, the rip is adapted to house at least one staple arrangement. Furthermore, in an embodiment, the rip is suitable to house completely or partially the at least one staple arrangement.

In an embodiment, the stapling structures are associated to the alignment structure by at least a coupling. The coupling can be any structure capable of performing the association between the stapling structure and the alignment structure. Furthermore, such coupling is able to provide the movement of the stapling structure along the alignment structure. In an embodiment, the association between the coupling and the alignment structure allows the movement of the coupling along the alignment structure. In an embodiment, the coupling comprises a channel or a hole that allows its movement along the alignment structure.

In an embodiment, the coupling is adapted in such a way to allow engaging and removing the stapling structure from the coupling and/or from the alignment structure.

In an embodiment, the surgical stapler of the present invention comprises at least a staple driver, where the staple driver is responsible for applying a force to at least a part of the staple arrangement, providing at least one stapling. A part of the staple arrangement means that the staple driver can apply a force: only on one segment of the staple arrangement; in many segments; progressively along the staple arrangement; or simultaneously in all staple arrangement.

The staple driver is able to execute the stapling of the staple arrangement, i.e. to apply a force to the staple arrangement, since this stapling can occur by many different ways. In some embodiments, the staple driver can be a mechanism that runs, in any direction, in the stapling structure, where it can be linearly or rotary actuated by actuators and housed in the stapling structure. In some embodiments, the actuator can be any mechanism comprising cable, engines, and pneumatic, hydraulic or electronic actuators. In an embodiment, the staple driver runs through at least one stapling structure in order to apply a force to the staple arrangement ejecting them from the rip.

In an embodiment, the stapling structures comprise a distal end with a convergent declination. The term “convergent declination” is understood as the distal ends of the stapling structures comprising an angular deviation, where such ends are converging to each other or to a common point, for example, by extending an imaginary line. The convergent declination distances the muscle from the aponeurosis, facilitating its introduction and guiding the stapling structure during a stapling procedure.

In a second aspect, the present invention discloses a surgical staple arrangement comprising at least a first part and a second part associable to each other comprising a plurality of clamping structure.

The clamping structure is any structure able to provide the association between the first and second part. This association can occur in many ways wherein the clamping structure has a one way movement, in another words, after executing a certain movement, it holds in a such position.

In an embodiment, the surgical staple arrangement is comprised by at least two clamping structures that are connected to each other.

In an embodiment, the clamping structures are collinearly connected to each other. The connection between the clamping structures forms a continuous staple arrangement, where clamping structures are, for instance, connected side by side. The arrangement of continuous staples allows distribution of forces applied on the staple arrangement when it is inside a patient, avoiding the release of the clamping structure.

In an embodiment, the clamping structures are connected by a flexible joint region. The flexible joint region allows the staple arrangement to bend over at least one axle. In an embodiment, the flexible joint region is formed by a narrowing of a material between the clamping structures.

In an embodiment, the clamping structures comprise a male staple and a female staple, in such a way that the male staple is associated to the female staple in order to associate the first part to the second part of the staple arrangement. In an embodiment, the first part comprises male staples and the second part comprises female staples. In another embodiment, the first part comprises female staples and the second part comprises male staples. In another embodiment, the male and female staple are mixed over the first and second part, in such a way that for each male staple a female staple is opposite arranged.

In an embodiment, the first and second parts of the surgical staple arrangement are bioabsorbable.

In an embodiment, the surgical stapler and the surgical staple arrangement as defined are used for stapling aponeurosis and/or for approximation of muscular or aponeurotic structures of the abdominal wall.

In another aspect, the present invention discloses a method for approximation of muscular or aponeurotic structures of the abdominal wall comprising the steps of:
a. locate at least one stapling structure in a muscular or aponeurotic structure of the abdominal wall;
b. approximation of muscular or aponeurotic structures of the abdominal wall;
c. stapling of the muscular or aponeurotic structures of the abdominal wall.

The stapling structure is any structure capable to be introduced in a muscular or aponeurotic structure of the abdominal wall, where said stapling structure is movable over an alignment structure. Thus, the stapling structure can be introduced in different types of muscular or aponeurotic structure of the abdominal wall of a patient, according needed.

In an embodiment, the stapling structures are located inside of the anterior rectal abdominal sheath.

In an embodiment, the stapling structures are located inside of the posterior rectal abdominal sheath.

In an embodiment, at least one stapling structure is located inside the anterior rectal abdominal sheath and at least one stapling structure is located inside the posterior rectal abdominal sheath.

In an embodiment, the approximation of muscular or aponeurotic structures of the abdominal wall comprises a rectilinear movement of the stapling structure along an alignment structure. This movement can be performed by a mechanism or by hand, and furthermore this movement can be performed manually or automatically.

In an embodiment, the step of stapling comprises triggering at least one staple driver that applies a force to at least a part of a staple arrangement providing stapling of the muscular or aponeurotic structures of the abdominal wall. The staple driver is able to eject at least a part of the staple arrangement from the stapling structure, in order to provide the stapling of the muscular or aponeurotic structures.

### EXAMPLE 1

### Surgical Stapler

In an embodiment, the surgical stapler comprises parallel stapling structures (1) attached to an alignment structure (2) by couplings (5), said couplings (5) comprising at least one channel associated to the alignment structure (2), wherein at least one coupling (5) is rectilinearly movable along the alignment structure (2). Said rectilinear movement allows the stapling structures (1) are parallel approximated or distanced providing linear junction of the muscular or aponeurotic structure.

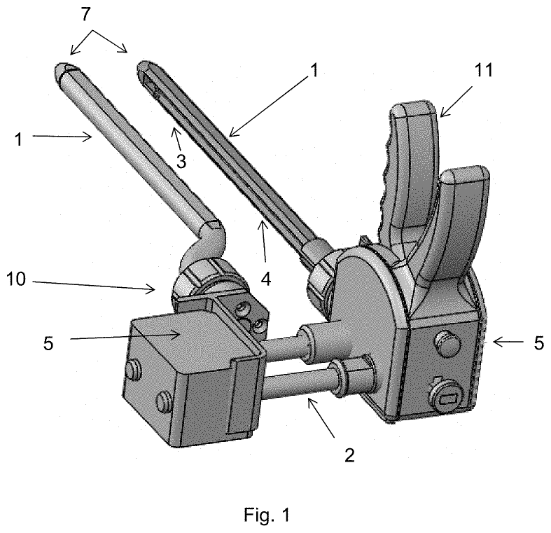

The alignment structure (2) comprises at least one shaft. In an embodiment, the alignment structure (2) is fixable attached to a first coupling (5) and a second coupling (5) is rectilinear movable along the alignment structure. In another embodiment, shown in the FIG. 1, the alignment structure (2) comprises two parallel shafts where the couplings (5) are associated, since this embodiment does not allow the stapling structures (1) to rotate over the shafts.

The coupling (5) comprises at least one channel for associating the alignment structure (2) and a connector (10) for engaging at least one stapling structure (1). In an embodiment, the coupling (5) is able to move rectilinearly along the alignment structure (2) and comprise a lock device, ensuring that the coupling (5) is fixable during the procedure.

In an embodiment, the stapling structure (1) comprises an elongated body with at least one rip (3), wherein the rip (3) hosts at least a part of a staple arrangement (4). In another embodiment, the stapling structures (1) comprise a length adjustment or a set of stapling structure with different lengths. Furthermore, in an embodiment, the stapling structures comprise a distal end with a convergent declination (7), where the distal ends of the stapling structures (1) comprising an angular deviation, since such ends are converging to each other or to a common point, for example, by extending an imaginary line, as clearly shown in FIG. 13.

In an embodiment, the surgical stapler comprises a staple driver (6) that is aligned with the rip (3) of at least one stapling structure (1). The staple driver (6), in its distal end, comprises a bevel (13) that is adapted to eject at least a staple of the staple arrangement (4). The staple driver (6) ejects the staple from the rip (3) of the stapling structure, compressing the staple arrangement (4), resulting its clamping.

In an embodiment, at least one coupling (5) comprises a trigger (11) that actuates the staple driver (6). A displacement mechanism housed in the coupling (5) is configured to introduce the staple driver (6) into the rip (3) of the stapling structure (1), by the pulling of the trigger (11).

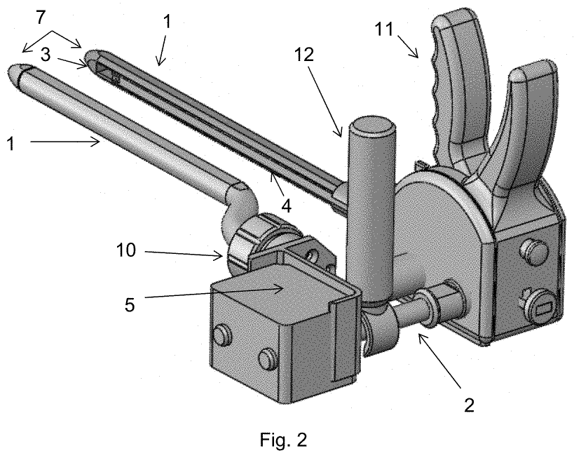

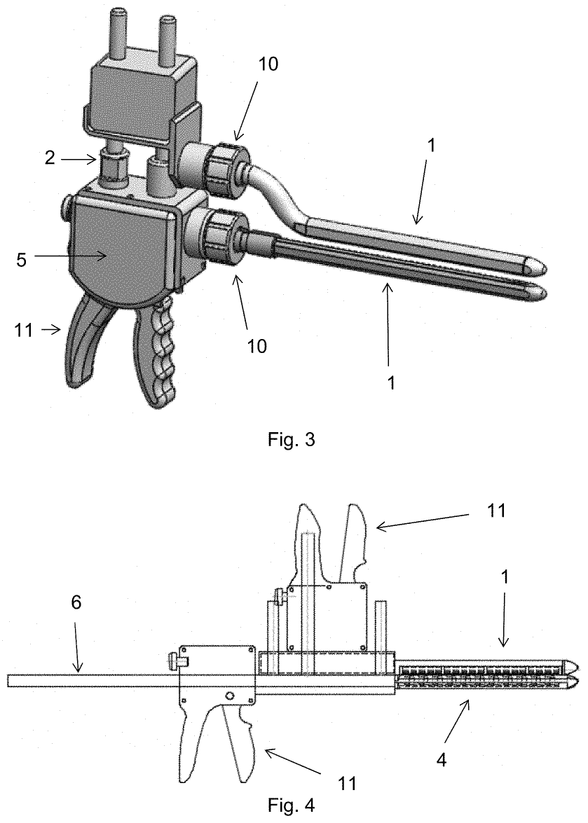

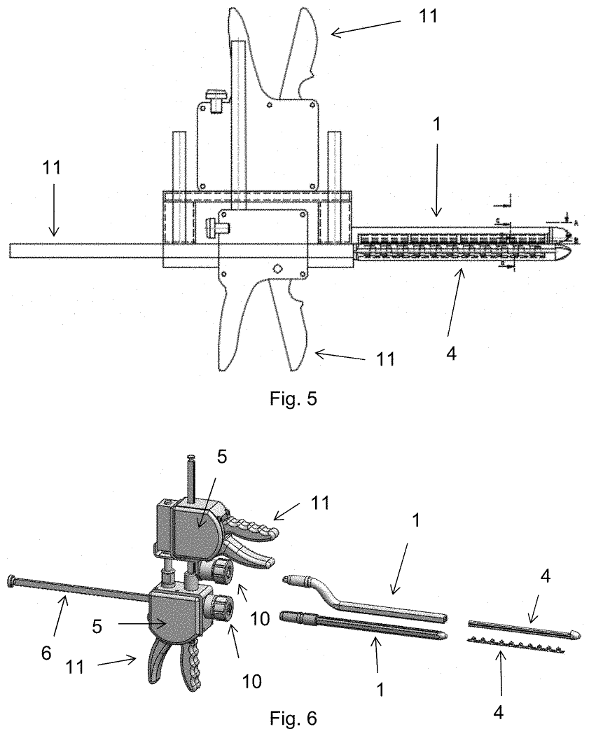

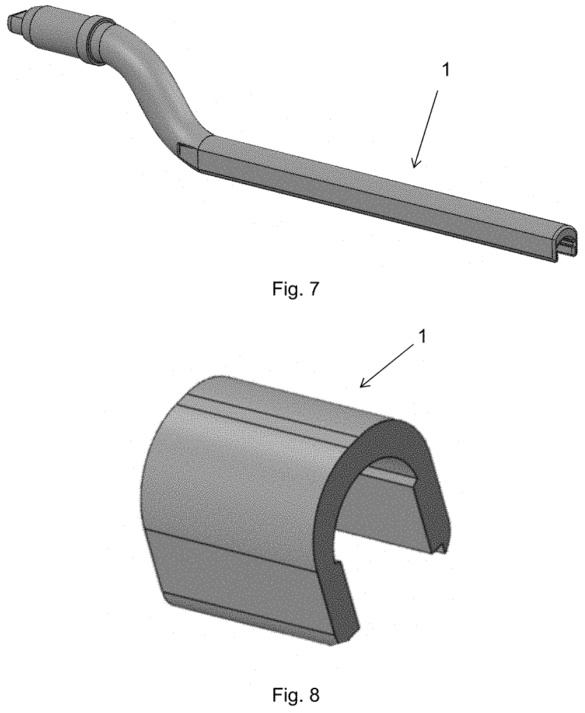

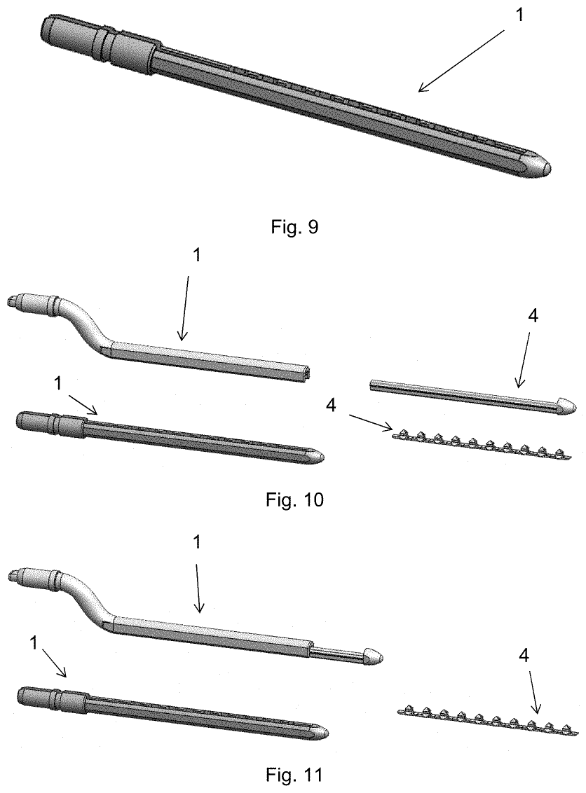

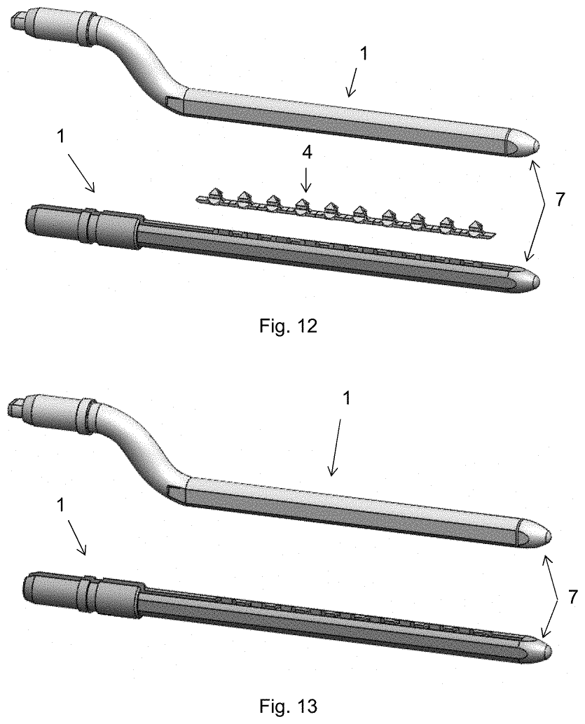

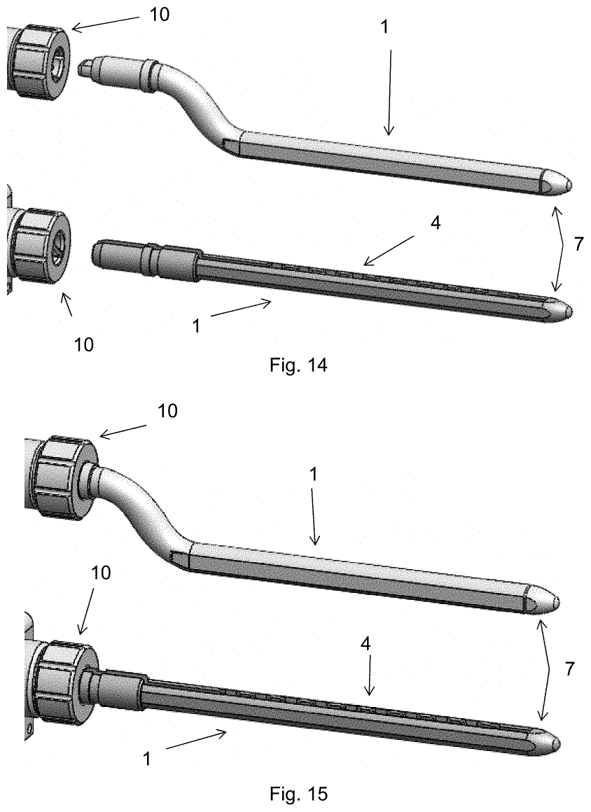

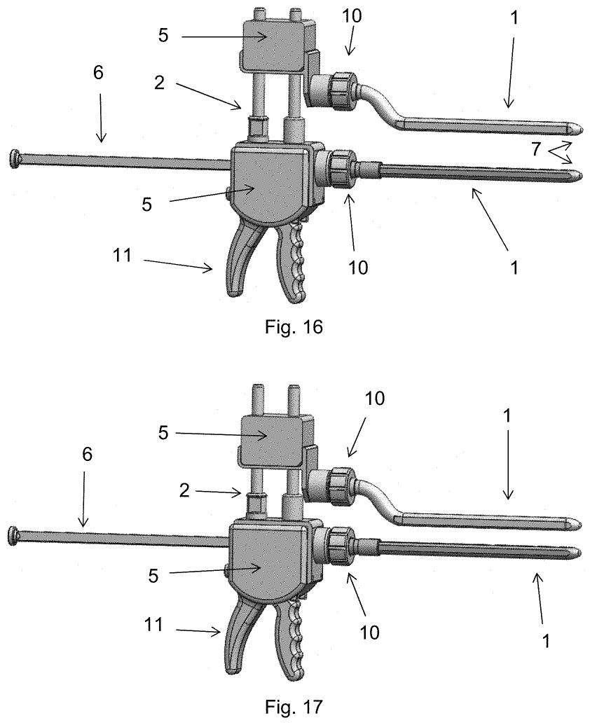

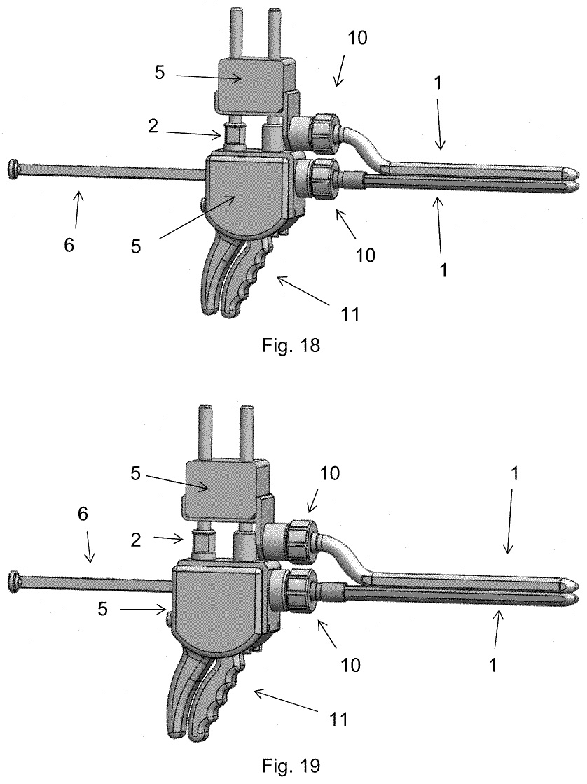

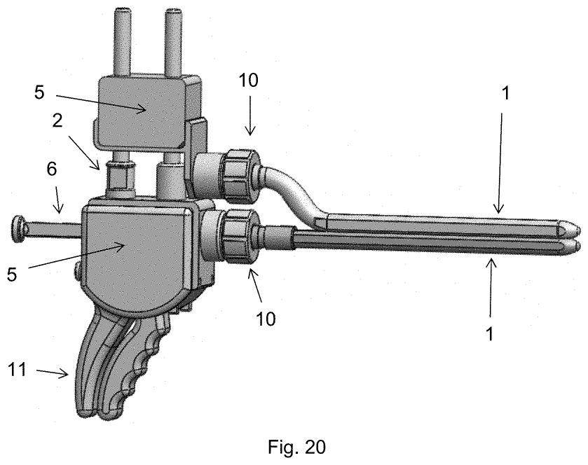

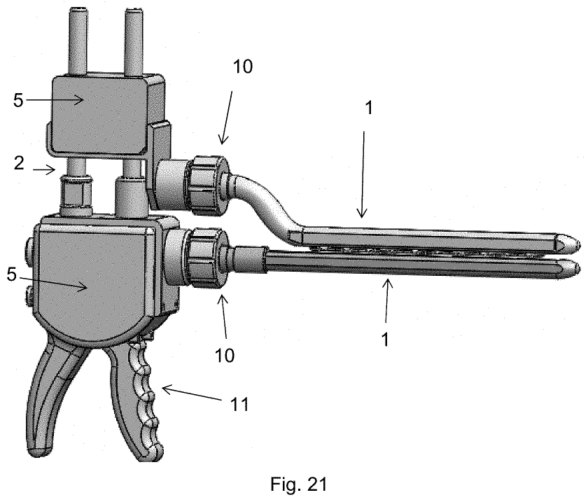

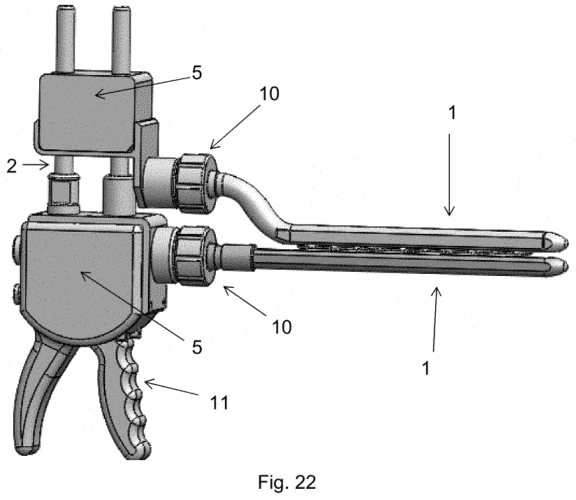

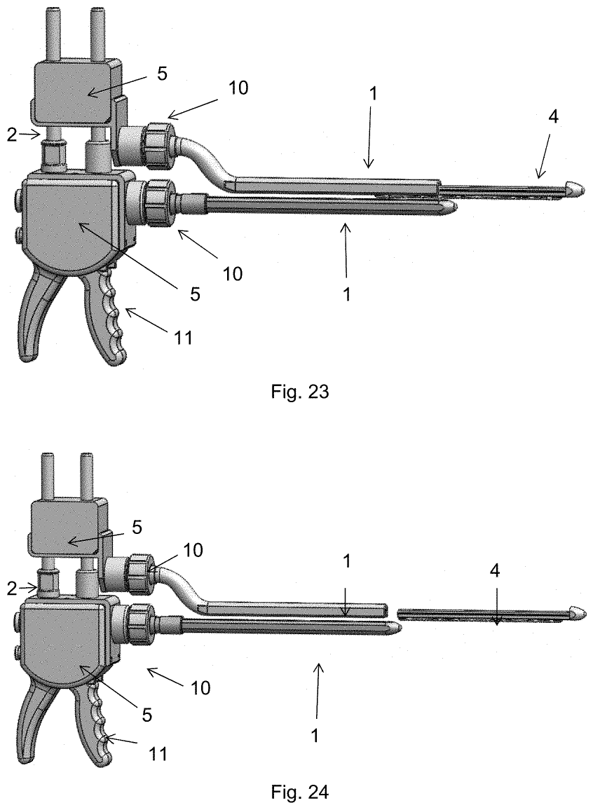

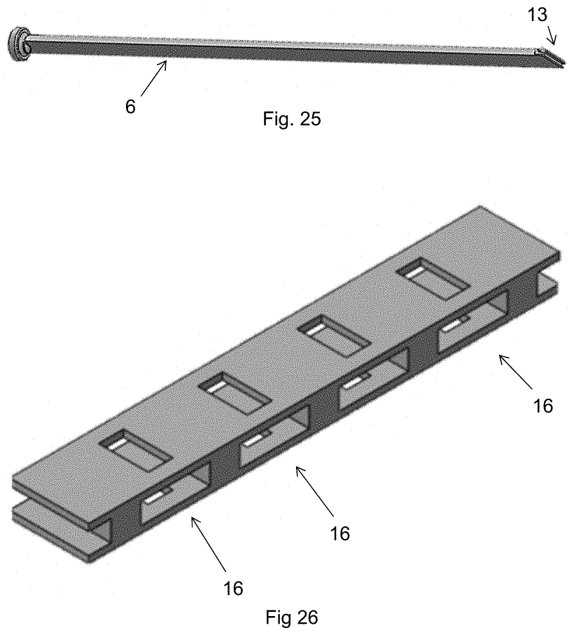

In an embodiment, as shown in FIG. 2, at least one coupling (5) comprises a handle (12). The handle (12) facilitates the handling of the stapler when moving the stapler and helps the control of the torsional force when pulling the trigger (11), it is important the stapler not to rotate during the clamping of the staple which could cause an injury or a clamping error.

In an embodiment, the staple arrangement (4) comprises at least a male staple and at least a female staple, wherein the male staple is opposite to the female staple.

In an embodiment, the alignment comprises two parallel shafts. In one distal end of the shafts, they are rigidly associated on a first coupling (5) and a second coupling (5) is movably associated to the shafts.

The first coupling (5) comprises a connector (10) for engaging a first stapling structure, a trigger (11) to actuate a staple driver (6), a hole for introducing the staple driver (6), two channels for associating the alignments structures.

The second coupling (5) comprises a connector (10) for engaging a second stapling structure, two channels for associating the alignment structures (2), a handle (12) for manipulate and move the coupling (5) along the alignment structure, a lock device to restrict its movement.

The hole of the first stapling structure comprises the same geometry as the staple driver (6), and then the hole guides the staple driver (6) without letting it rotate.

Depending on the dimension of the connectors (10), the stapling structure (1) can comprise a curved segment allowing the stapling structures (1) to get close enough while the connectors (10) still spaced.

Both stapling structures (1) comprise rips (3) to host a part of a staple arrangement (4). The difference is that the rip (3) of one stapling structures (1) is made to host a male staple and the other is made to host a female staple. In an embodiment, where the staples are connected in a rail arrangement the rips (3) comprise a format to ensure that the male staple stay aligned to the female staple.

### EXAMPLE 2

### Staple Arrangement Examples

In an embodiment, the surgical staple arrangement (4) comprising at least a male staple and at least a female staple, wherein the male staple comprises an elongated body (14) and a head (15), said head (15) larger than the elongated body (14), and the female staple is a frame structured comprising a perimeter adapted to receive the male staple.

The construction of the male staple permits its introduction in the perimeter of the female staple, but restricts its return. This is due to the head (15) of the male staple comprises a pyramidal shape, the sides of the head (15) deforms the perimeter of the female staple, allowing the male staple enter the perimeter, but once the base of the head (15) pass through the perimeter it cannot go back because the perimeter returns to a no deformed state. It therefore allows the staples are associated to each other after the procedure.

In an embodiment, the female staple comprises a rail arrangement formed by a plurality of female staples, wherein each female staple is collinear positioned by a flexible joint (9). The unions of the staples create a more rigid structure to resist the clamping force and the retraction of the aponeurosis, notwithstanding the flexible joint (9) allows the staple to bend according to muscular movements.

In an embodiment, the male staple comprises a rail arrangement formed by a plurality of male staples, wherein each male staple is collinear positioned by a flexible joint (9). The unions of the staples create a more rigid structure to resist the clamping force and the retraction of the aponeurosis, notwithstanding the flexible joint (9) allows the staple to bend according to muscular movements.

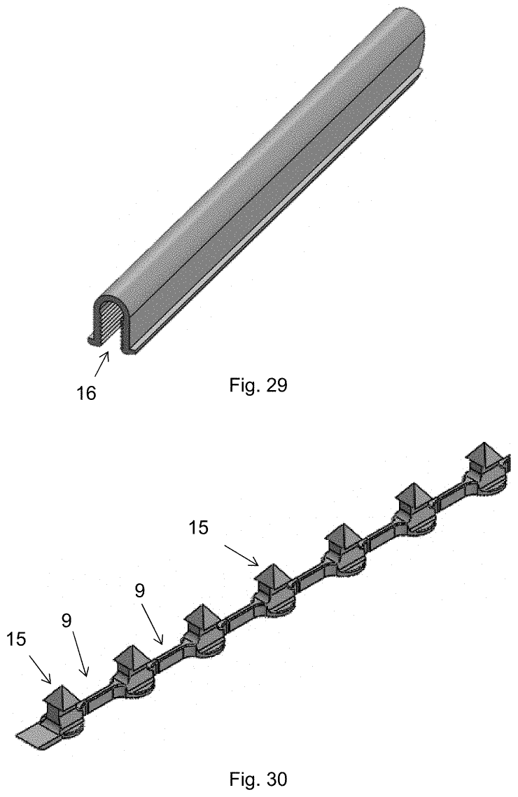

In an embodiment, as shown in FIGS. 29 and 53 the female staple comprises an elongated “U” format, with serrated inside walls.

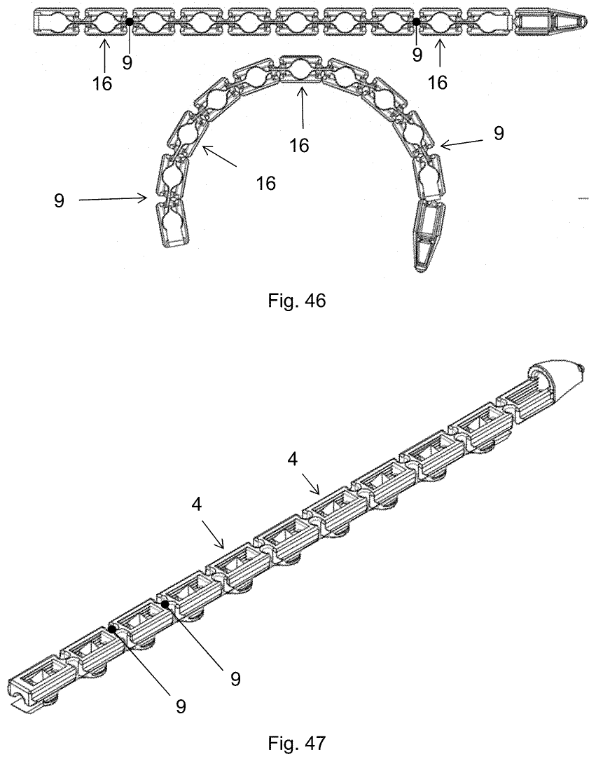

In an embodiment, the female staple comprises a rail arrangement formed by a plurality of frame structures (16) connected by a flexible joint (9). In a preferred embodiment, the flexible joints (9) are thinner than the frame structure (16), allowing the rail arrangement to bend, as shown in FIG. 46.

The female staple can be closed in one side or comprise both sides opened.

In an embodiment the male and female staples are configured to be introduced in a rip (3) of a stapling structure (1) of the surgical stapler.

In an embodiment, the male staple comprises a square based pyramid head (15), where the base is larger than the elongated body (14), creating a sudden change of dimension. In others embodiments, the base of the pyramidal head (15) can also comprise any geometry.

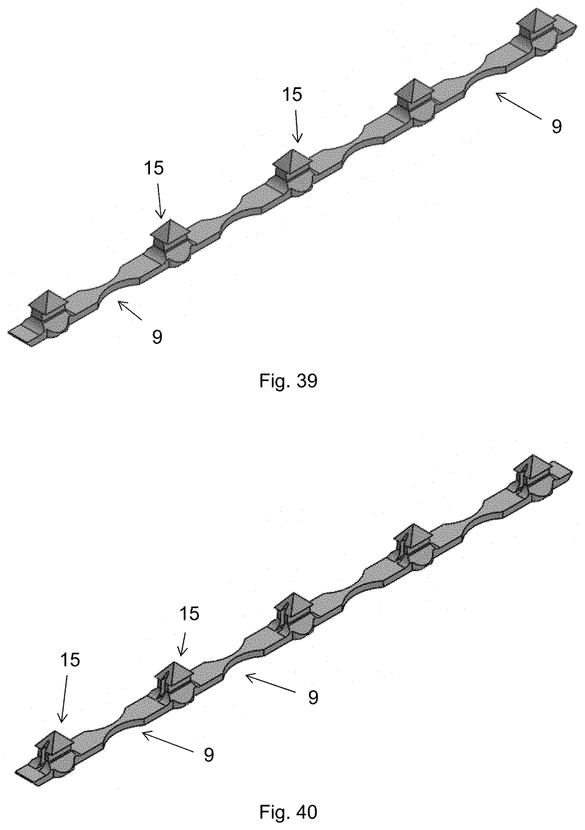

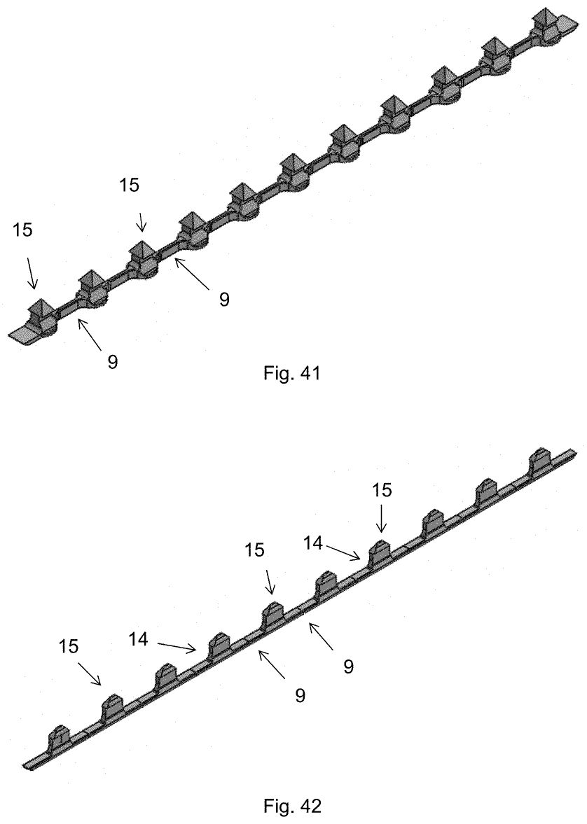

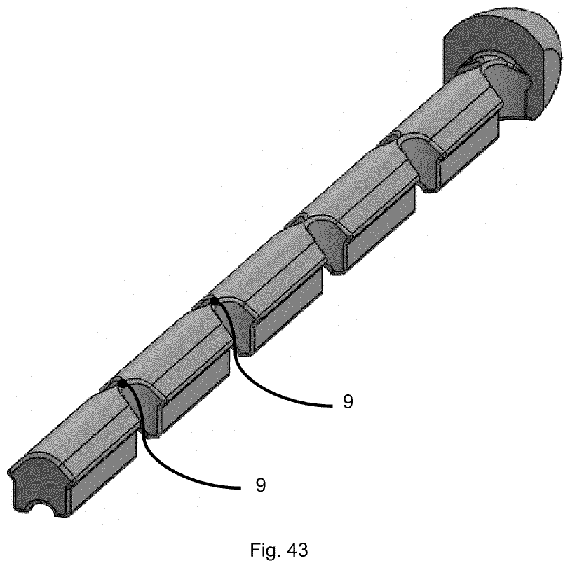

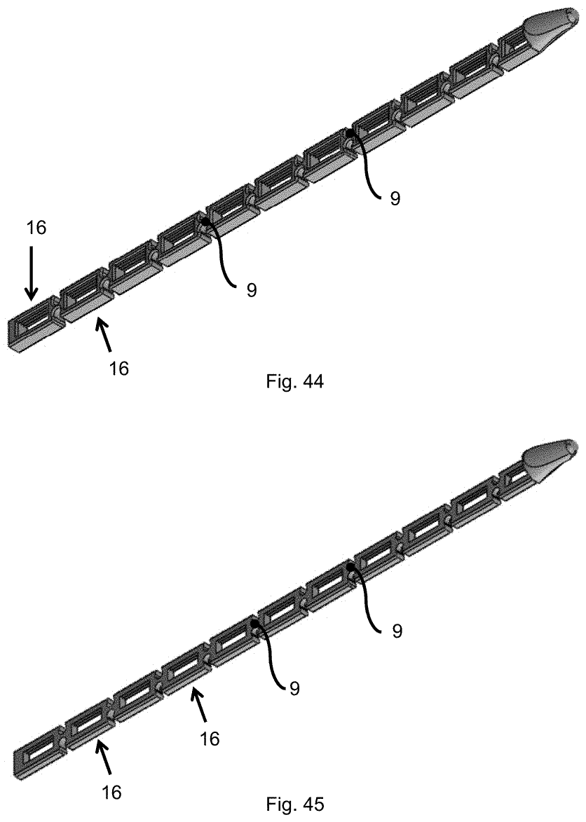

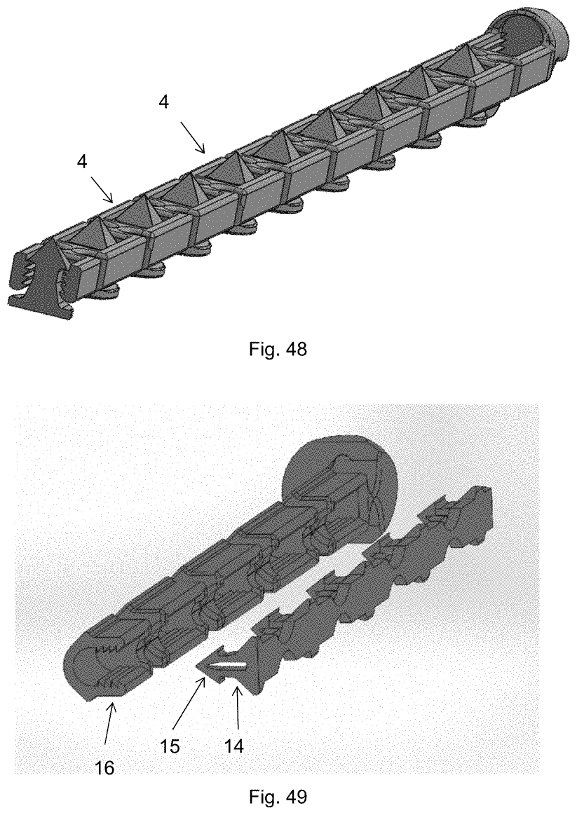

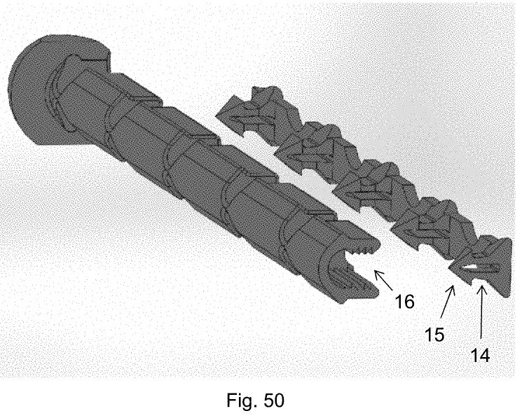

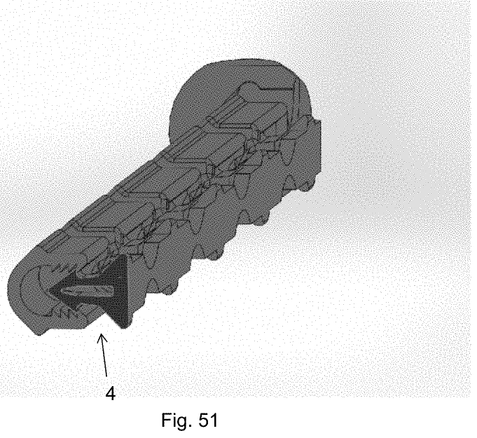

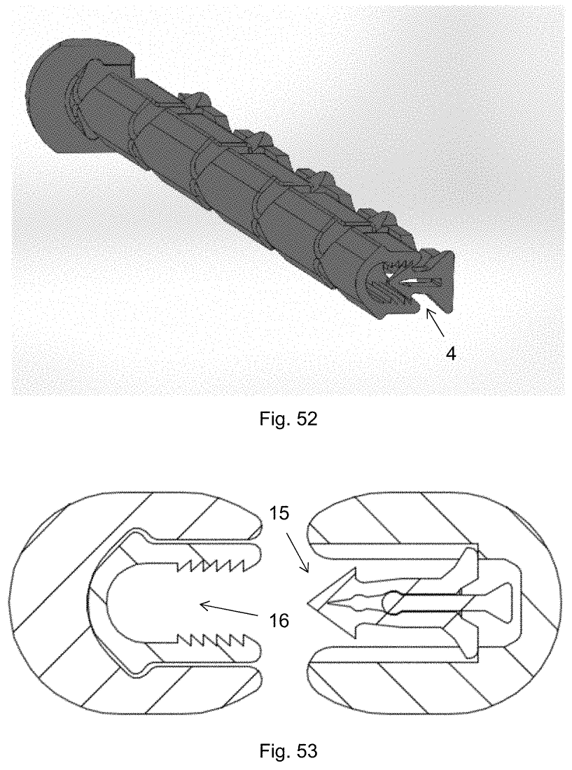

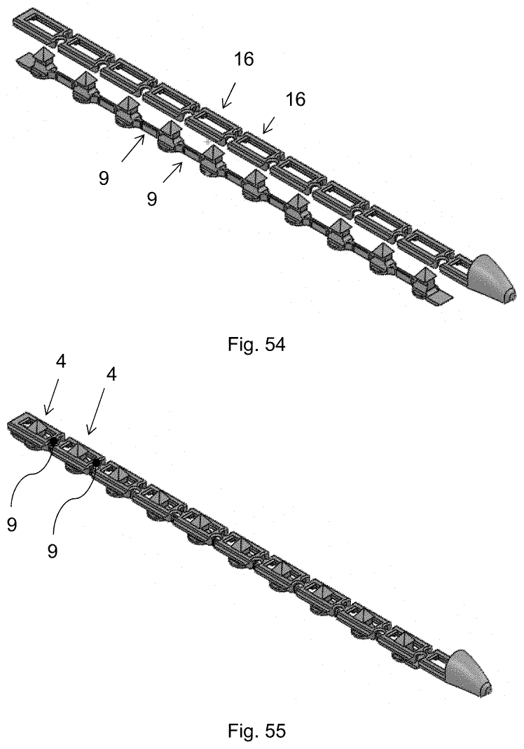

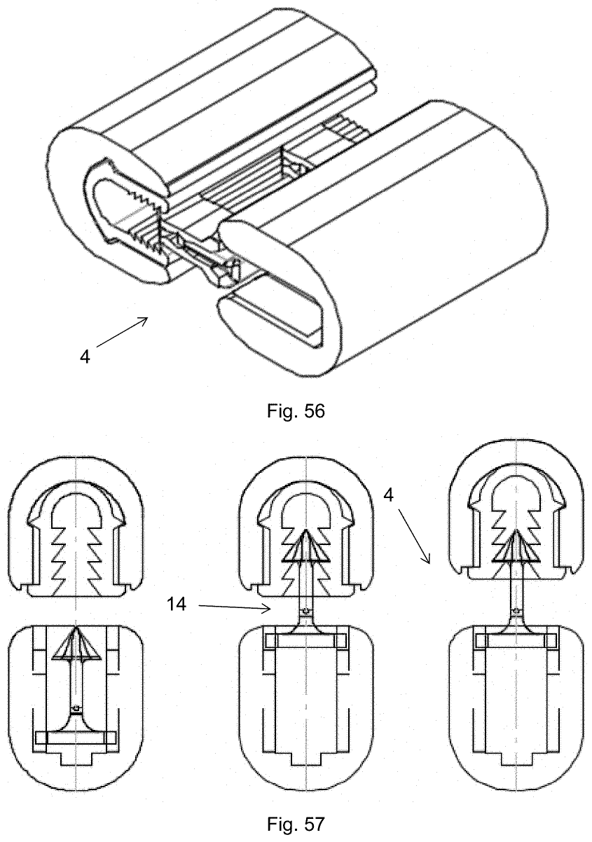

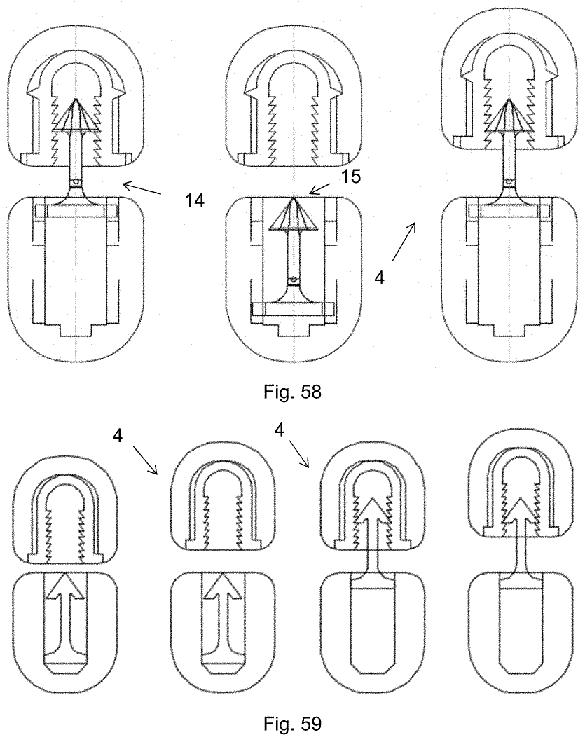

In an embodiment, as shown in FIG. 30, the male staple comprises a rail arrangement formed by a plurality of male staples connected by a flexible joint (9). In a preferred embodiment, the flexible joints (9) are thinner than the elongated body (14), allowing the rail arrangement to bend, as shown in FIGS. 39 to 50.

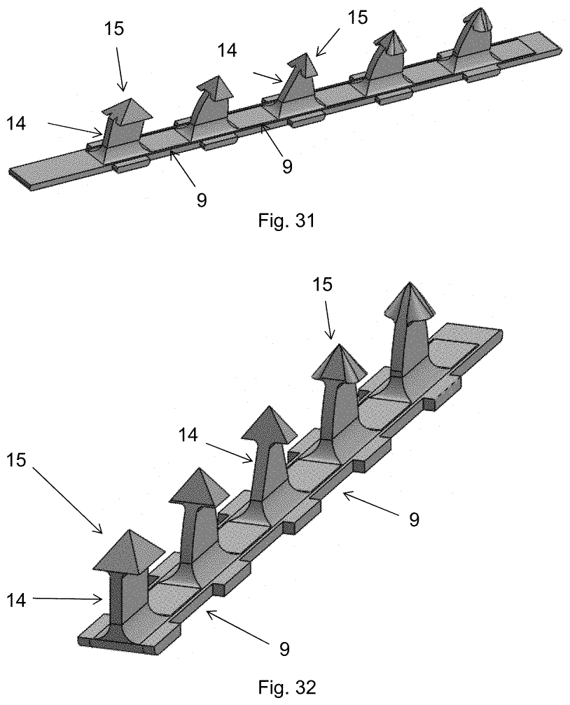

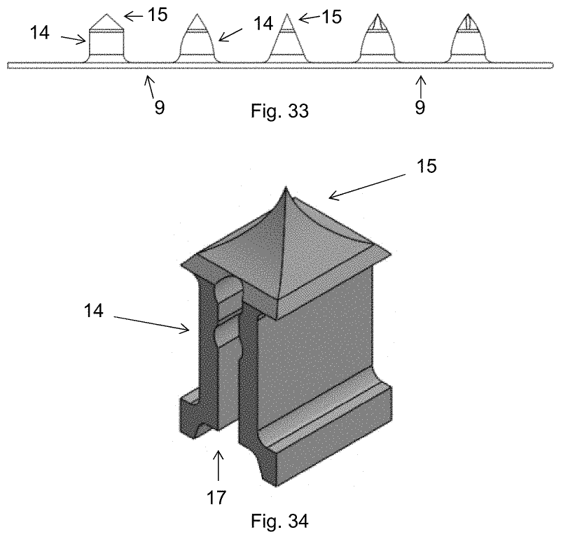

In an embodiment, staples comprising different geometry can be connected to form a rail arrangement, as shown in FIGS. 31 and 32.

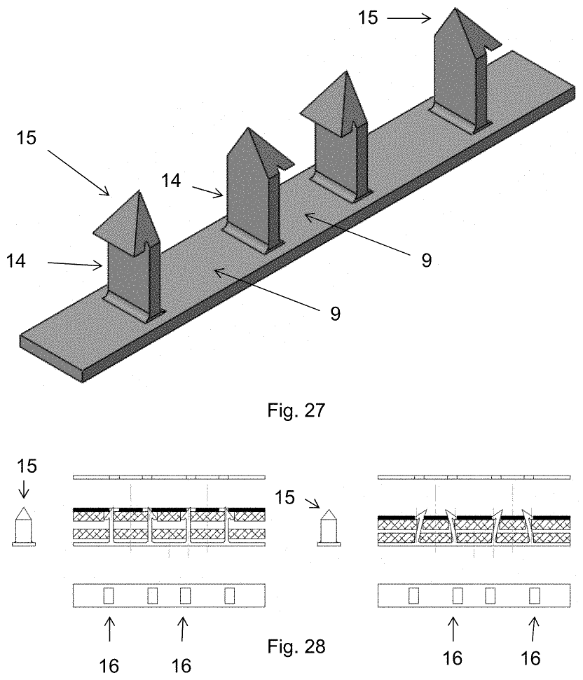

In an embodiment, as shown in FIGS. 26 to 28, the male staple comprises a half pyramidal head (15) associated to an elongated body (14), wherein in the rail arrangement the staples are distributed in pair, positioned back to back. The female staple in a rail arrangement comprises holes misaligned with the head (15) of the male staple, thus when applying a clamp force, the inclined wall of the pyramid head (15) in touch with the hole cause the bend of the elongated body (14), than when the head (15) passes through the hole, the elongated body (14) contract back to position, locking the male staple to the female staple.

In an embodiment, the edges of the pyramid head (15) can comprise rounded edges, as shown in FIG. 32.

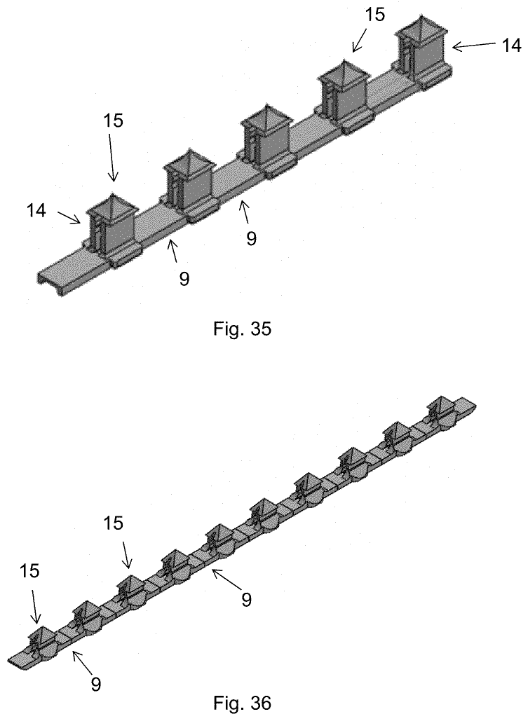

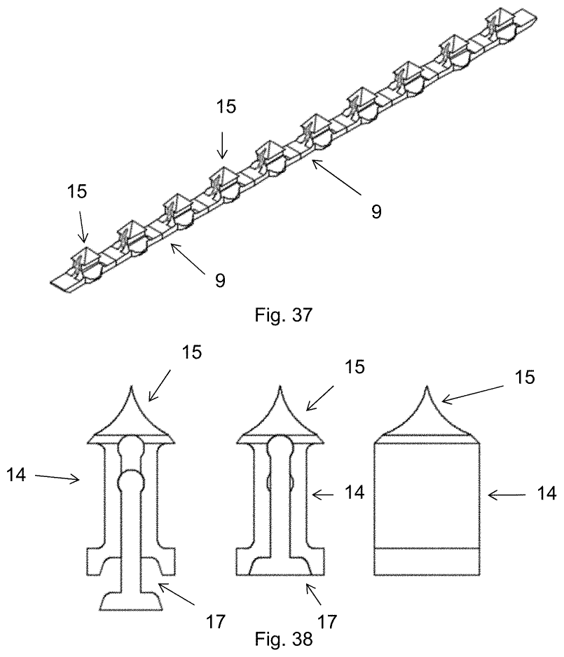

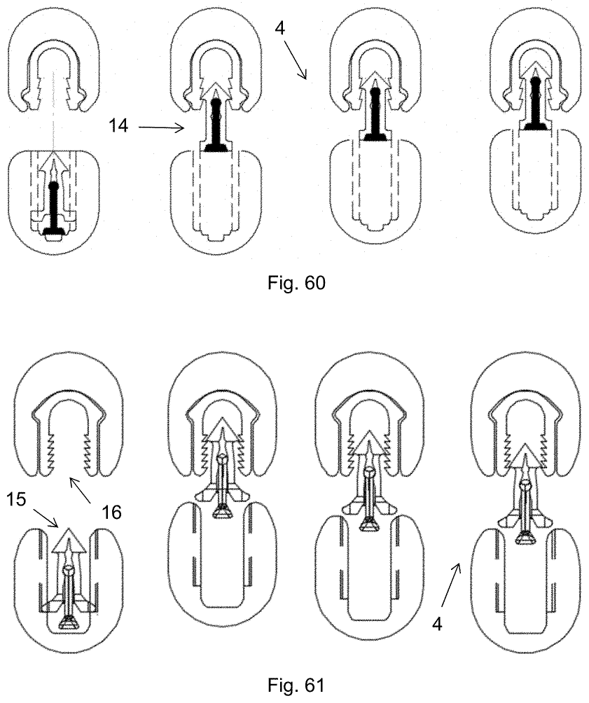

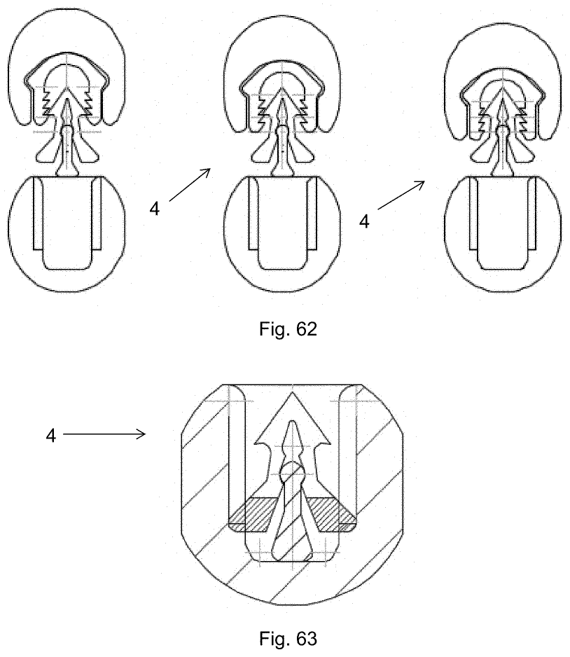

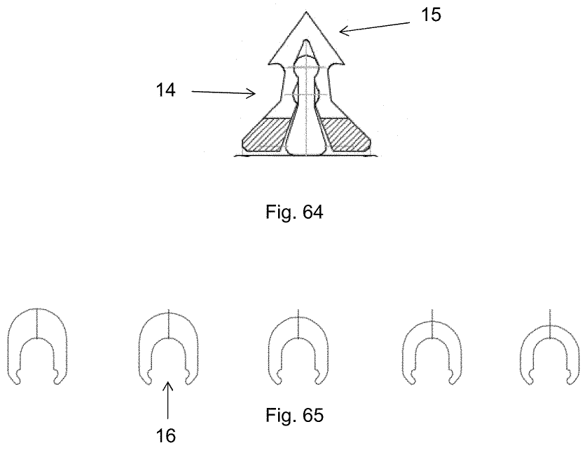

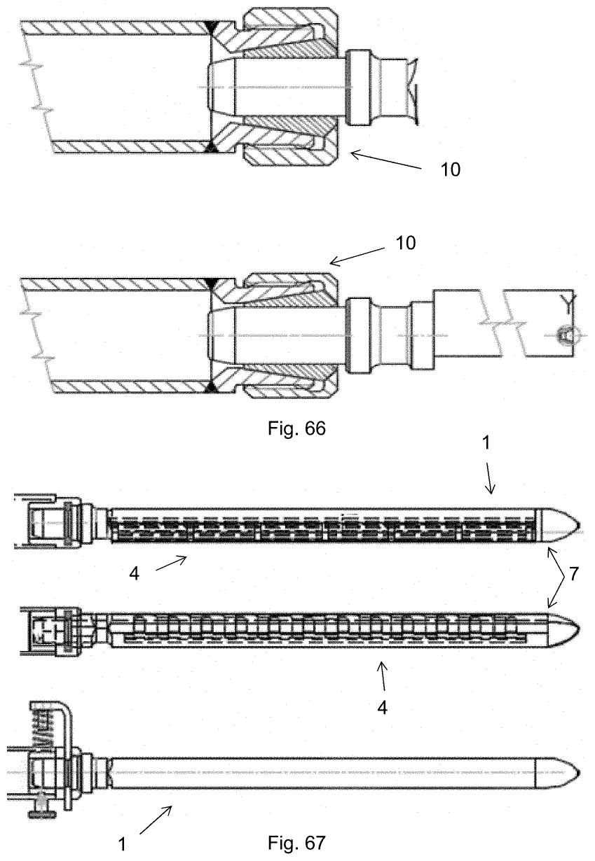

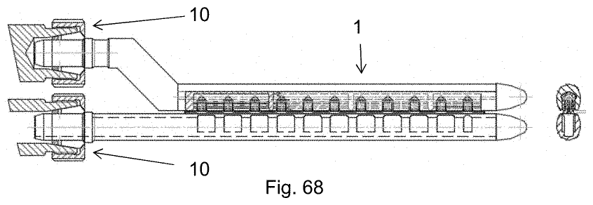

In an embodiment, the male staple comprises an internal channel where is inserted a locking pin (17). Before clamping, the locking pin (17) stays in a retracted position, not filling all the internal channel, this allow the head (15) of the male staple to deform while is introduced in a female staple, after the male staple is clamped in the female staple, the locking pin (17) advance and fill all the internal channel, restricting the head (15) to deform, thus maintaining the staple arrangement (4) clamped. This embodiment is shown in FIGS. 34 to 38 and the FIGS. 60 to 63 shows this embodiment housed in the stapling structure (1), also showing some steps of the clamping movement.

In an embodiment, the staple arrangement (4) is covered by antibiotics.

In an embodiment, the staple arrangement (4) is made of bioabsorbable material.

### EXAMPLE 3

### Method for Stapling Aponeurosis

In an embodiment, the method for stapling aponeurosis comprises the steps of:
a. locate the first stapling structure (1), associable to a first coupling (5), in a first part of the aponeurosis;
b. locate the second stapling structure (1), associable to a second coupling (5), in a second part of the aponeurosis;
c. approach the stapling structures (1) by moving a coupling (5) rectilinearly; and
d. operate a trigger (11), moving a staple driver (6) that eject at least one part of a staple arrangement (4).

The method for stapling aponeurosis is used to approximate aponeurosis that is stretched or ruptured. Thus, a first stapling structure (1) is positioned in one side of the aponeurosis and a second stapling structure (1) is positioned in the other side of the aponeurosis, so that a portion of the aponeurosis stays between both stapling structures (1). Then, one of the stapling structures (1) is connected to its coupling (5) and the approach of the stapling structure (1) is done. The approximation put the stapling structures (1) and the aponeurosis together, in consequence, approximating the muscles.

With the stapling structures (1) together, the trigger (11) is operated, moving the staple driver (6) into the rip (3) of the stapling structure (1) hosting the male staple, the distal end of the staple driver (6) comprises a bevel (13), which when moved against the male staple, the contact between the bevel (13) and the male staple ejects the male staple against the female staple, causing its clamping.

In an embodiment, the length of the stapling structures (1) is adjustable or there are stapling structures (1) with different lengths, so a step of adjusting the lengths of the stapling structures (1) or selecting the right stapling structure (1) is necessary. The length of the stapling structure (1) should be long enough to reach the patient's xiphoid.

To initiate the procedure the stapling structures (1) must be loaded with the staple arrangement (4), one stapling structure (1) is disengaged from its coupling (5). Then, with the incisions made on the patient's body, the stapling structures (1) are inserted through the incisions, the distal end of the stapling structures (1) separate the muscle from the aponeurosis, leaving only a portion of aponeurosis between both stapling structures (1). With the stapling structures (1) in position, the disengaged stapling structure is engaged back to its coupling (5), and by the rectilinear movement of one coupling (5) along the alignment structure the stapling structures (1) are approximated. After the approach of the stapling structures (1), the staple driver (6) is triggered, running in the rip (3), by a displacement mechanism, against the male staple, ejecting the male staple from the stapling structure, the male staple is ejected by the interaction of the bevel (13), comprised on the distal end of the staple driver (6), with the male staple.

When ejected, the male staple is forced against the female staple causing the clamping of the staple arrangement (4). With the staple arrangement (4) clamped, the stapling structures (1) can be pulled out of the patient's body, leaving the staple arrangement (4) clamped in position.

Those well versed in the art will value the knowledge here, and may reproduce the invention in the manner provided and other variants, covered within the scope of appended claims.

1. A surgical stapler comprising stapling structures associated to an alignment structure.

2. The surgical stapler according to claim 1, wherein at least one stapling structure comprises a movement along the alignment structure.

3. The surgical stapler according to claim 2, wherein the movement comprises distancing and approximating of the stapling structures along the alignment structure.

4. The surgical stapler according to claim 2, wherein the stapling structures is rectilinearly movable along the alignment structure.

5. The surgical stapler according to claim 4, wherein the stapling structures are associated to the alignment structure by at least a coupling.

6. The surgical stapler according to claim 1, wherein the stapling structures comprise at least one rip.

7. The surgical stapler according to claim 6, wherein the rip is adapted to contain at least one staple arrangement.

8. The surgical stapler according to claim 7, wherein it comprises at least a staple driver that applies a force to at least a part of staple arrangement providing at least one stapling.

9. The surgical stapler according to claim 1, wherein the stapling structures comprise a distal end comprising convergent declination.

10. A surgical staple arrangement comprising at least a first part and a second part associable to each other comprising a plurality of clamping structure.

11. The surgical staple arrangement according to claim 10, wherein at least two clamping structures are connected to each other.

12. The surgical staple arrangement according to claim 11, wherein the clamping structures are collinearly connected to each other.

13. The surgical staple arrangement according to claim 12, wherein the clamping structures are connected by a flexible joint region.

14. The surgical staple arrangement according to claim 10, wherein the clamping structures comprise a male staple and a female staple.

15. The surgical staple arrangement according to claim 10, wherein said first part and second part are bioabsorbable.

16. A method for stapling aponeurosis comprising steps performed by a surgical stapler comprising stapling structures associated to an alignment structure and a surgical staple arrangement as defined in claim 10.

17. A method for approximation of muscular or aponeurotic structures of the abdominal wall comprising:
a. locate at least one stapling structure in a muscular or aponeurotic structure of the abdominal wall;
b. approximation of muscular or aponeurotic structures of the abdominal wall; and
c. stapling of the muscular or aponeurotic structures of the abdominal wall.

18. The method according to claim 17, wherein the stapling structures are located inside of the anterior rectal abdominal sheath.

19. The method according to claim 17, wherein the stapling structures are located inside of the posterior rectal abdominal sheath.

20. The method according to claim 17, wherein the stapling structures are located inside of the anterior and posterior rectal abdominal sheath.

21. The method according to claim 17, wherein the approximation comprises a rectilinearly movement of the stapling structure along an alignment structure.

22. The method according to claim 17, wherein the stapling comprises triggering of a staple driver applying a force to at least a part of a staple arrangement providing stapling of the muscular or aponeurotic structures of the abdominal wall.
